(19) United States
(12) Patent Application Publication
(10) Pub. No.: US 2012/0323086 A1
(43) Pub. Date: Dec. 20, 2012

(54) ALARM SENSITIVITY CONTROL FOR PATIENT MONITORS

(75) Inventor: Bryan Hansen, Mead, CO (US)

(73) Assignee: Nellcor Puritan Bennett LLC, Boulder, CO (US)

(21) Appl. No.: 13/163,975

(22) Filed: Jun. 20, 2011

(51) Int. Cl. A61B 5/02; A61B 5/00; A61B 5/01; A61B 5/024; A61B 5/08; A61B 5/021; G08B 23/00; A61B 5/145

(52) U.S. Cl. 600/301; 340/573.1; 600/300; 600/364; 600/481; 600/529; 600/485; 600/549

(57) ABSTRACT

Physical monitoring systems are disclosed which may allow for alarm sensitivity adjustment. A user may indicate an alarm sensitivity of a patient monitoring system to a physiological parameter, signal metric, operating condition metric, or other parameter or metric. The patient monitoring system may configure one or more alarm settings based on the indicated alarm sensitivity. Low sensitivity may reduce the probable occurrence or severity of alarm activations, while high sensitivity may increase the probable occurrence or severity of alarm activations.

The present disclosure relates to alarm sensitivity control, and more particularly relates to providing a range of alarm sensitivities for a patient monitor.

### SUMMARY

A patient monitoring system is provided with adjustable alarm sensitivity. The patient monitoring system may receive a user indication of alarm sensitivity, and may configure one or more alarm settings based on the user indication. The patient monitoring system may configure alarm settings for physiological parameters (e.g., blood oxygen saturation, pulse rate, respiration rate, blood pressure, temperature), operating condition metrics (e.g., power level, sensor signal quality, patient movement, sensor movement), signal metrics (e.g., waveform pattern detection), any other suitable parameter or metric, or any combination thereof. Alarm settings may include alarm limit thresholds (e.g., for values, change, rate of change, or trend), durations of limit violations, number of limit violations, integral thresholds (e.g., time integrals of signal excursions outside of a limit), any other suitable settings, or any combination thereof. Alarm sensitivity may be indicated by selection of a preset based on user identification, patient identification, medical procedure, drug administration, default preset, any other suitable preset, or any combination thereof.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system, in accordance with some embodiments of the present disclosure;

FIG. 2 is a block diagram of the illustrative patient monitoring system of FIG. 1 coupled to a patient, in accordance with some embodiments of the present disclosure;

FIG. 3 shows a table of illustrative parameters for which an alarm may be activated, in accordance with some embodiments of the present disclosure;

FIG. 4 shows an illustrative display of a physiological monitoring system, in accordance with some embodiments of the present disclosure;

FIG. 5 shows an illustrative panel of two plots, each including a time series and alarm limits, in accordance with some embodiments of the present disclosure;

FIG. 6 is a flow diagram of illustrative steps for configuring alarm settings of a patient monitoring system based at least in part on a user indication, in accordance with some embodiments of the present disclosure;

FIG. 7 is a flow diagram of illustrative steps for configuring alarm settings of a patient monitoring system, in accordance with some embodiments of the present disclosure;

FIG. 8 is a flow diagram of illustrative steps for configuring alarm settings of a patient monitoring system to an indicated alarm sensitivity, in accordance with some embodiments of the present disclosure;

FIG. 9(a) shows an illustrative indication of alarm sensitivity, in accordance with some embodiments of the present disclosure;

FIG. 9(b) shows an illustrative indication of alarm sensitivity using user identification, in accordance with some embodiments of the present disclosure;

FIG. 9(c) shows an illustrative indication of alarm sensitivity using a patient identification, in accordance with some embodiments of the present disclosure; and

FIG. 9(d) shows an illustrative indication of alarm sensitivity using a mode, in accordance with some embodiments of the present disclosure.

### DETAILED DESCRIPTION OF THE FIGURES

The present disclosure is directed towards methods and systems for configuring alarm sensitivity. In some embodiments, a patient monitoring system may configure alarm settings to provide a particular alarm sensitivity. The patient monitoring system may include an oximeter, or other suitable device, or combinations thereof, for monitoring physiological activity of a patient.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate and blood pressure.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=I_{O}(λ)exp(−(sβ_{O}(λ)+(1−s)βᵣ(λ))l(t)) (1)

where:
λ=wavelength;
t=time;
I=intensity of light detected;
I₀=intensity of light transmitted;
s=oxygen saturation;
β₀,βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log I_{O}−(sβ_{O}+(1−s)βᵣ)l. (2)

2. Eq. 2 is then differentiated with respect to time to yield

$$\frac{d log I}{d t} = - ( s {β}_{O} + ( 1 - s ) β r ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{O} ( {λ}_{R} ) + ( 1 - s ) {β }_{r} ( {λ}_{R} )}{s {β}_{O} ( {λ}_{IR} ) + ( 1 - s ) {β }_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{O} ( {λ}_{IR} ) - {β}_{r} ( {λ}_{IR} ) ) - \\ \frac{d log I ( {λ}_{IR} )}{d t} ( {β}_{O} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for s using the relationship of Eq. 5 yields

$$s = \frac{{β}_{r} ( {λ}_{R} ) - R {β}_{r} ( {λ}_{IR} )}{R ( {β}_{O} ( {λ}_{IR} ) - {β}_{r} ( {λ}_{IR} ) ) - {β}_{O} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{\frac{d I}{d t}}{I} , \qquad ( 10 )$$

Eq. 8 becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R ,\end{matrix} \qquad ( 11 )$$

which defines a cluster of points whose slope of y versus x will give R when

x=[I(t₂,λ_{IR})−I(t₁,λ_{IR})]I(t₁,λR), (12)

and

y=[I(t₂,λ_{IR})−I(t₁,λ_{IR})]I(t₁,λR). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

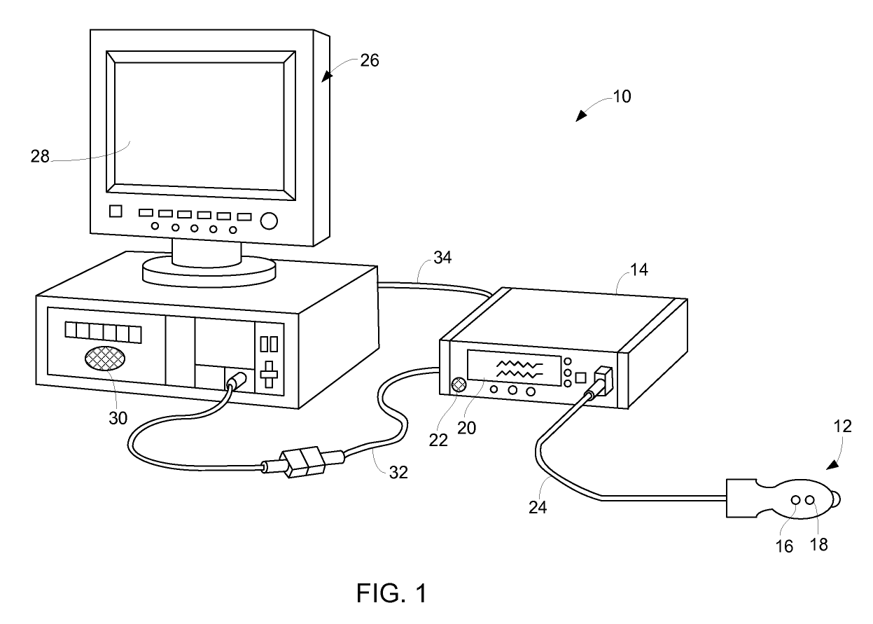

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) which may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According to another embodiment, system 10 may include a plurality of sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In an embodiment, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood pressure, blood oxygen saturation) based at least in part on data relating to light emission and detection received from one or more sensor units such as sensor unit 12 and an additional sensor. In an alternative embodiment, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the monitor 14 includes a blood pressure monitor. In some embodiments, the system 10 includes a stand-alone blood pressure monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24.

In the illustrated embodiment, system 10 includes a multi-parameter patient monitor 26. The monitor 26 may include a cathode ray tube display, a flat panel display (as shown) such as a liquid crystal display (LCD) or a plasma display, or may include any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood oxygen saturation generated by monitor 14 (referred to as an “SpO₂” measurement), pulse rate information from monitor 14 and blood pressure from monitor 14 on display 28. Multi-parameter patient monitor 26 may include a speaker 30.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

Calibration device 80, which may be powered by monitor 14 via a cable 82, a battery, or by a conventional power source such as a wall outlet, may include any suitable signal calibration device. Calibration device 80 may be communicatively coupled to monitor 14 via cable 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

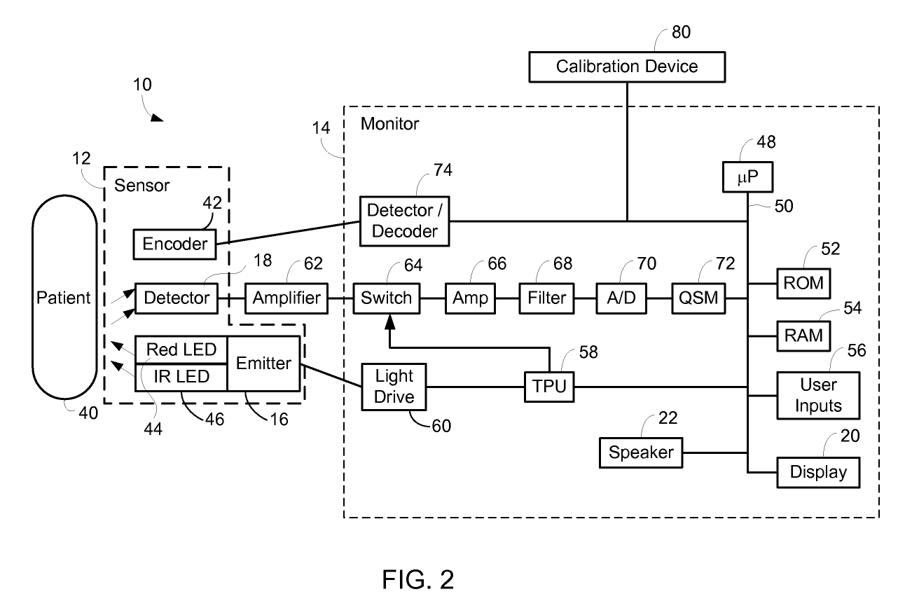

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In one embodiment, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor emits only a Red light while a second emits only an IR light. In another example, the wavelengths of light used are selected based on the specific location of the sensor.

It will be understood that, as used herein, the term “light” may refer to energy produced by radiation sources and may include one or more of ultrasound, radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which, for example, blood pressure and other measurements may be determined based at least in part on the signal or signals received at sensor unit 12. For example, some pulse oximetry sensors rely on equations to relate an area under a portion of a photoplethysmograph (PPG) signal corresponding to a physiological pulse to determine blood pressure. These equations may contain coefficients that depend upon a patient's physiological characteristics as stored in encoder 42. Encoder 42 may, for instance, be a coded resistor which stores values corresponding to the type of sensor unit 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor unit 12; the wavelengths of light emitted by emitter 16; the particular wavelength each sensor in the sensor array is monitoring; a signal threshold for each sensor in the sensor array; any other suitable information; or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 fills up. In one embodiment, there may be multiple separate parallel paths having components equivalent to amplifier 66, filter 68, and/or A/D converter 70 for multiple light wavelengths or spectra received.

In an embodiment, microprocessor 48 may determine the patient's physiological parameters, such as SpO₂, pulse rate, and/or blood pressure, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable the microprocessor to determine the thresholds based at least in part on algorithms or look-up tables stored in ROM 52. In some embodiments, user inputs 56 may be used enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values which may generally apply to the patient, such as, for example, age ranges or medication families, which the user may select using user inputs 56. In some embodiments, display 20 may exhibit one or more selectable options such as alarm settings, alarm sensitivity, or both which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals or otherwise identify noise components in order to prevent them from affecting measurements of physiological parameters derived from the sensor signals.

It will be understood that the present disclosure is applicable to any suitable signal and that PPG signals, are used merely for illustrative purposes. Those skilled in the art will recognize that the present disclosure has wide applicability to other signals including, but not limited to, other biosignals (e.g., electrocardiograms, electroencephalograms, electrogastrograms, electromyograms, pulse rate signals, pathological signals, ultrasound signals, any other suitable biosignals), dynamic signals, non-destructive testing signals, condition monitoring signals, fluid dynamic signals, geophysical signals, astronomical signals, electrical signals, financial signals, sound and speech signals, chemical signals (e.g., arising from chemical kinetics), meteorological signals (e.g., climate signals), any other suitable signals, or any combination thereof.

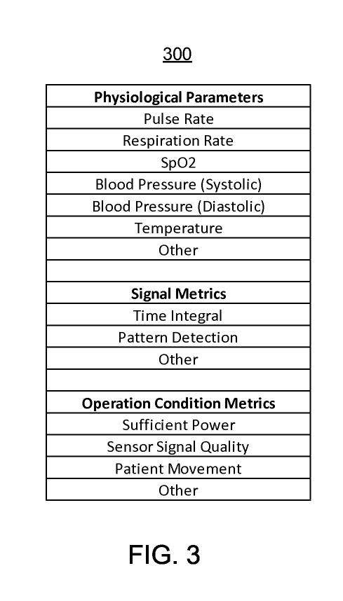

FIG. 3 shows a table 300 of illustrative physiological parameters, signal metrics, and operating condition metrics, in accordance with some embodiments of the present disclosure. A patient monitoring system (e.g., patient monitoring system 10 of FIG. 1) may be configured to provide alarms, warnings or other indicators based at least in part on one or more alarm settings. In some embodiments, a patient monitoring system (e.g., patient monitoring system 10 of FIG. 1) may be configured to provide alarms, warnings or other indicators based at least in part on the value of a signal (e.g., a voltage), physiological parameter such as, for example, SpO2, pulse rate, respiration rate, blood pressure, changes in any of the foregoing, computed quantities of any the foregoing, trends of the foregoing, any other suitable basis for providing an alarm, or any combination thereof. In some embodiments, a patient monitoring system may be configured to provide alarms, warnings or other indicators based at least in part on signal metrics such as, for example, plethysmograph waveform pattern, time integral of signal excursion outside of a limit, any other suitable signal metric for providing an alarm, or any combination thereof. In some embodiments, a patient monitoring system may be configured to provide alarms, warnings or other indicators based at least in part on operating condition metrics such as, for example, sufficient power (e.g., sensor power delivered), sensor signal quality (e.g., sensor signal strength, signal noise level), patient and/or sensor movement, any other suitable operating condition metric for providing an alarm, or any combination thereof.

In some embodiments, suitable hardware may be used to communicate an activated alarm such as, for example, speaker 22 (e.g., an alert tone, a beep warning), display 20 (e.g., a displayed warning, a flashing alarm color), ROM 52 (e.g., a saved warning message, recorded data), any other suitable hardware, or any combination.

Alarms, warnings, and other indicators may be based on a comparison of a suitable value with a limit threshold (e.g., a value less than a lower limit), comparison of a slope or change with a change threshold (e.g., a rate of change greater than a rate threshold), a trend (e.g., value increasing, first derivative decreasing), a duration that an alarm condition is achieved (e.g., a value greater than a high limit for a particular amount of time), an integral of a deviation of a time series with a suitable alarm limit (e.g., as shown in FIG. 5), a number of limit violations (e.g., over a time interval), any other suitable comparison or determination, or any combination thereof.

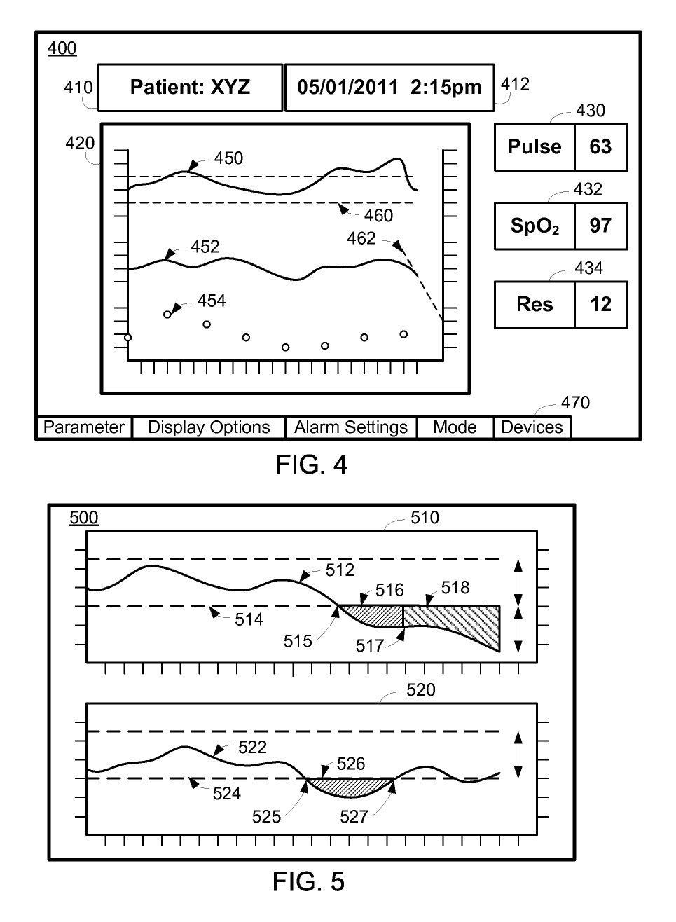

FIG. 4 shows an illustrative display 400 of a physiological monitoring system, in accordance with some embodiments of the present disclosure. Display 400 may include any suitable physiological information, identifying information, alarm information, any other suitable information, or any combination thereof. As illustratively shown in FIG. 4, display 400 may include time series (e.g., time series 450, 452, and 454 arranged in panel 420), alphanumeric readouts (e.g., text boxes 430, 432, and 434), identification information (e.g., text box 410 showing a patient ID), selectable options (e.g., option bar 470), any other suitable text, graphic, image, or information (e.g., text box 412), or any combination thereof. Illustrative display 400 includes time series of pulse rate (time series 450), SpO₂ (time series 452), and respiration rate (time series 454), although any suitable values, time series, related information (e.g., trend, slope, average, metrics derived thereof), or combinations thereof may be displayed.

In some embodiments, display 400 may include alarm limits or other indicators. For example, display 400 may include a high limit, low limit, or both, for a physiological parameter as shown illustratively by alarm limits 460 for pulse rate time series 450 in FIG. 4. If a suitable value of pulse rate (e.g., instantaneous value, moving average value, ensemble average value, previous value, value at a discrete time) falls outside of the high and low limits, an alarm condition may be satisfied and an alarm may be activated. In a further example, rate of change limit 462 may be displayed by display 400. Although alarm limits are shown in illustrative display 400 for clarity, alarm limits and conditions need not be displayed.

It will be understood that the abscissa of the plot shown in panel 420 may be time, sample number, value number, or other index which may track the progression of a value. It will also be understood that the ordinate(s) of the plot shown in panel 420 may be indexed in any suitable unit or measure, referenced to any suitable origin, normalized, or otherwise mathematically scaled to provide an indication of the absolute or relative value of a particular time series.

Violations of alarm settings may include excursions of a parameter or metric outside of a nominal range of values or states, possibly under a set of additional conditions. For example, alarm violations may include exceeding a limit threshold (e.g., high or low limit, slope limit), switching to a new state (e.g., system power switched off versus on, sensor uncoupled to patient monitor versus coupled to the patient monitor), exceeding a number threshold of limit violations, exceeding a duration threshold of a limit violation, any other suitable violation of an alarm settings, or any combination thereof.

In an illustrative example, the pulse of a patient may be monitored by a patient monitoring system rate in beats per minute (BPM). Alarm settings for pulse rate may include high and low alarm limits of 75 and 50 BPM, and a limit of ±5 BPM/minute for changes in pulse rate. In some embodiments, if the monitored pulse rate of the patient increases above 75 BPM (i.e., alarm limit violation), decreases below 50 BPM (i.e., alarm limit violation), or changes by more than 5 BPM/minute (i.e., alarm limit violation) in either direction, an alarm may be activated. In some embodiments, an alarm may only be activated if the alarm limit violation persists for a particular time interval (e.g., 10 seconds or any other time interval). Any suitable parameter or metric may be monitored for an alarm limit violations.

FIG. 5 shows a panel 500 of illustrative plots 510 and 520 which include respective time series 512 and 522, and corresponding alarm limits 514 and 524, in accordance with some embodiments of the present disclosure. Alarm limits 514 and 524 are shown to include both a high limit and a low limit, although both need not be used. At point 515 of FIG. 5, time series 512 is shown to decrease below the low limit of alarm limits 514. Shaded region 516 shows the integrated region corresponding to the integral of the deviation of time series 512 from the lower limit over time until point 517 (i.e., the shaded area between the curves). Eq. 14 shows an illustrative integral calculation, in which the difference D(t) between the time series S(t) and a threshold value V(t) is integrated over time from t0 to T (e.g., referencing FIG. 5, time of point 515 to time of point 517 or other suitable point subsequent to point 515). The threshold value V(t) may, but need not, vary with time. Additionally, although D(t) is shown as an absolute value in Eq. 14, D(t) may be positive or negative depending upon the type of alarm limit. In some embodiments, calculations other than a difference may be used to quantify a deviation of a time series outside of an alarm limit. Eq. 15 shows a numerical approximation to an integral over n data points, although any suitable quadrature, integral, summation, other suitable calculation, or combination thereof may be used (e.g., trapezoid rule, Simpson's rule). Any suitable calculation may be used, instead of or in addition to an integral, to quantify the excursion of a parameter outside of an alarm limit.

I=∫ₜ₀^{T}|S(t)−V(t)|dt=∫ₜ₀^{T}D(t)dt (14)

I=Σᵢ₌₁ⁿ(Dᵢ)Δtᵢ (15)

A set of various illustrative conditional expressions are shown in Eq. 16. If an integral (e.g., I of Eqs. 14 or 15) is less than a threshold integral value I₀, then no alarm may be activated. If an integral is greater than I₀ and the duration of the alarm limit violation is greater than a limit L, then an alarm may be activated. If the absolute value of the deviation D is above a threshold x, then an alarm may be activated. The illustrative alarm conditions shown in Eq. 16 are presented as exemplary conditions, and it will be understood that any suitable conditions, or combinations thereof, may be used to determine whether to activate an alarm.

$$[ \begin{matrix}if I < {I}_{0} , then do not activate alarm \\ if I > {I}_{0} \& ( T - {t}_{0} ) < L , then activate alarm \\ if \vert D \vert > x , then activate alarm\end{matrix} ] \qquad ( 16 )$$

In some embodiments, an integral such as shaded region 516 may be compared with an alarm threshold value, and if the integral exceeds the threshold, an alarm is activated. Such an alarm setting factors both the amplitude and duration of deviation and may allow alarm activations based on small amplitude deviations, or “spikes” over short time scales, to be avoided. For example, the alarm threshold value may be reached at point 517, at which point the alarm is activated, as shown by the different shading of shaded region 518 relative to shaded region 516. The alarm may stay activated as the deviation continues as shown by shaded region 518. In some embodiments, if the amplitude of the deviation decreases to a particular threshold, the alarm may be deactivated. Any suitable conditions may be used to reset or otherwise deactivate an alarm. As shown in plot 520, time series 522 decreases below the low limit of alarm limits 524 at point 525, and the integration begins, as shown by shaded region 526. At point 527, prior to the alarm threshold being reached, time series 522 no longer deviates from the lower alarm limit, and the integration ceases.

In some embodiments, alarm activation may depend on more than one alarm setting. For example, referring to plot 510, alarm activation may depend on the integration of the deviation between time series 512 and lower alarm limit of alarm limits 514, shown by shaded regions 516 and 518, and on the magnitude of the deviation. If both the integral and the magnitude of the deviation reach respective alarm thresholds, then an alarm is activated. If only one alarm threshold (or no alarm threshold) is reached, then no alarm is activated. An alarm setting may have any suitable number of conditions, and may require any suitable number of these conditions to be met to activate the alarm.

In some circumstances, it may be desirable to adjust or otherwise manage the sensitivity of a physiological monitor to an alarm. The sensitivity of a physiological monitor to an alarm may be adjusted or otherwise managed by a user, a physiological monitoring system, a processing facility, any other suitable entity, or any combination thereof. For example, the sensitivity of a physiological monitor may be adjusted by a clinician depending upon the expected propensity of an emergency situation for a particular patient. In a further example, the sensitivity of a physiological monitor may be reduced to reduce the likelihood of false alarm activations (e.g., due to instantaneous spikes or other transient deviations). In a further example, the sensitivity of a physiological monitor to one or more alarm limits of one or more physiological parameters may be adjusted depending upon a medical procedure.

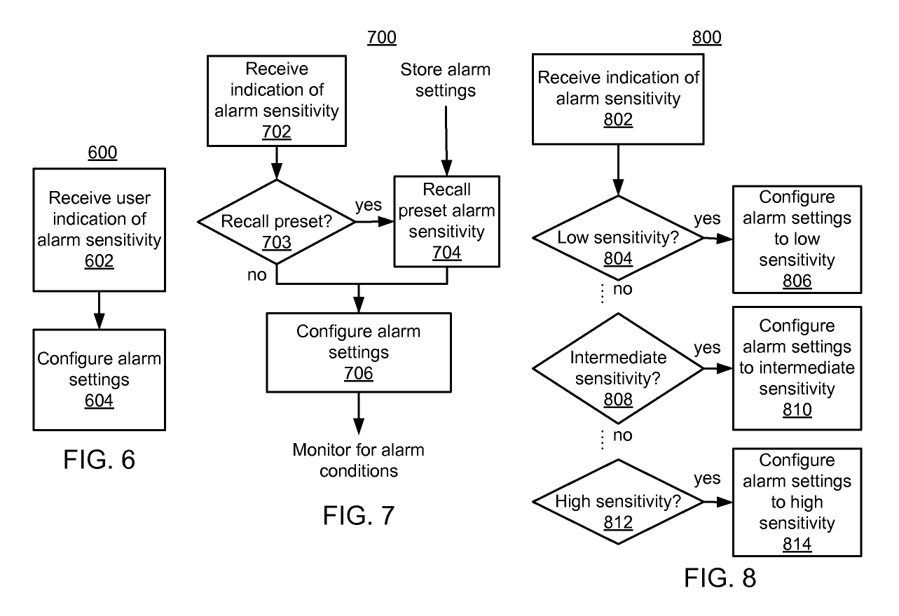

FIG. 6 is a flow diagram 600 of illustrative steps for configuring alarm settings of a patient monitoring system based at least in part on a user indication, in accordance with some embodiments of the present disclosure.

Step 602 may include receiving a user indication of one or more alarm sensitivities. The user indication may be received by a patient monitor to a user input interface (e.g., user inputs 56 of FIG. 2) which may include a device such as, for example, a keyboard, keypad (e.g., hard command buttons), mouse (e.g., with on-screen cursor), touchscreen (e.g., soft command buttons), microphone (e.g., voice activated control), any other suitable user input device which may be part of or coupled to a patient monitoring system, or any combination thereof. For example, step 602 may include a user entering a number (e.g., a number from 1 to 100 with 100 being most sensitive) on a keypad to indicate a desired alarm sensitivity. In a further example, step 602 may include a user selecting an on-screen option (e.g., a slide-bar, a pull-down menu, a check box, a text field) using a mouse or touchscreen command to indicate a desired alarm sensitivity. In a further example, step 602 may include a user selecting one alarm sensitivity option from a plurality of alarm sensitivity options (e.g., using an on-screen highlighted region to highlight the desired option).

Step 604 may include configuring alarm settings based at least in part on the received user indication of step 602. Alarm settings may include alarm limits (e.g., thresholds), parameters which may activate an alarm, alarm reset conditions, the type of alarm functionality to provide (e.g., audible, visible, event marking), any other suitable alarm settings, or any combination thereof. Configuring an alarm setting may include increasing or decreasing one or more alarm limits, selecting one or more parameters (e.g., physiological parameters, operating condition metrics) to be monitored for an alarm condition, determining under what conditions an alarm is to be reset, determining how data is to be stored, determining how data is to be displayed, updating the alarm settings in memory, storing the updated alarm settings in memory, any other suitable action related to configuring an alarm setting, or any combination thereof.

FIG. 7 is flow diagram 700 of illustrative steps for configuring alarm settings of a patient monitoring system, in accordance with some embodiments of the present disclosure. In some embodiments, alarm settings may be configured based on a user indication, preset alarm sensitivity settings, or both.

Step 702 may include receiving a user indication of one or more alarm sensitivities. The user indication may be received by a patient monitor to a user input interface which may include a device, such as, for example, a keyboard, keypad, mouse, touchscreen, microphone, any other suitable user input device which may part of or coupled to a patient monitoring system, or any combination thereof. For example, step 702 may include a user entering a number on a keypad to indicate a desired alarm sensitivity. In a further example, step 702 may include a user selecting an on-screen option using a mouse or touchscreen command to indicate a desired alarm sensitivity. In a further example, step 702 may include a user selecting one alarm sensitivity option from a plurality of alarm sensitivity options.

In some embodiments, a user indication may include determining whether to recall a previously set (“preset”) alarm sensitivity, as shown by step 703. In some embodiments, a user indication may provide a direct indication of a desired sensitivity, and need not recall a preset alarm sensitivity. In some embodiments, a user may indicate that a preset alarm sensitivity is desired at step 703. The determination of whether to recall a preset alarm sensitivity may be performed by the user, or by processing equipment of a patient monitoring system.

Step 704 may include recalling a previously set (“preset”) alarm sensitivity. Recalling a preset alarm sensitivity may include recalling preset alarm limits, which parameter(s) which may activate an alarm, alarm reset conditions, the type of alarm functionality to provide (e.g., audible, visible, event marking), recalling any other suitable stored alarm settings, or any combination thereof. In some embodiments, alarm settings may be stored (e.g., for later use) in memory. In some embodiments, a set of stored alarm settings may be categorized or otherwise arranged according to user (e.g., a clinician), patient, medical procedure (e.g., surgical procedure, drug administration), any other suitable designation, or any combination thereof. In some embodiments, step 704 may be performed in response to the user indication of step 702. For example, a user may indicate that a particular preset alarm sensitivity is to be used. In some embodiments, step 704 need not receive a user indication in order to be performed (i.e., step 702 need not be performed). For example, a patient monitor may recall a preset alarm sensitivity as a default sensitivity in the absence of user input.

Step 706 may include configuring alarm settings based at least in part on the received user indication of step 702, based at least in part on the recalled preset alarm sensitivity of step 704, or both. Alarm settings may include alarm limits (e.g., thresholds), parameters which may activate an alarm, alarm reset conditions, the type of alarm functionality to provide (e.g., audible, visible, event marking), any other suitable alarm settings, or any combination thereof. Configuring an alarm setting may include increasing or decreasing one or more alarm limits, selecting one or more parameters (e.g., physiological parameters, operating condition metrics) to be monitored for an alarm condition, determining under what conditions an alarm is to be reset, determining how data is to be stored, determining how data is to be displayed, updating the alarm settings in memory, storing the updated alarm settings in memory, any other suitable action related to configuring an alarm setting, or any combination thereof.

In some embodiments, one or more parameters (e.g., physiological parameters, operating condition metrics) may be monitored for conditions which violate one or more of the configured alarm settings of step 706.

FIG. 8 is a flow diagram 800 of illustrative steps for configuring alarm settings of a patient monitoring system to an indicated alarm sensitivity, in accordance with some embodiments of the present disclosure.

Step 802 may include receiving a user indication of one or more alarm sensitivities. The user indication may be received by a patient monitor to a user input interface (e.g., user inputs 56 of FIG. 2) which may include a device such as, for example, a keyboard, keypad, mouse, touchscreen, microphone, any other suitable user input device which may be part of or coupled to a patient monitoring system, or any combination thereof. For example, step 802 may include a user entering a number on a keypad to indicate a desired alarm sensitivity. In a further example, step 802 may include a user selecting an on-screen option using a mouse or touchscreen command to indicate a desired alarm sensitivity. In a further example, step 802 may include a user selecting one alarm sensitivity option from a plurality of alarm sensitivity options.

Depending upon the alarm sensitivity (e.g., low, high, intermediate) indicated by the user in step 802, alarm settings may be configured differently. As shown by illustrative steps 804, 808, and 812 of FIG. 8, a range of alarm sensitivities may be available. Accordingly, a range of configurations may be available, as shown by steps 806, 810, and 814 of FIG. 8. Although shown as discrete determinations in FIG. 8, any of steps 804, 808, and 812 may be performed in any suitable order, simultaneously, to the exclusion of other steps, as a yes-no determination, as a table look-up, as a single step, by any other suitable determination, or any combination thereof. In some embodiments, the range of alarm sensitivities may be divided into any suitable number of intervals. For example, the range may be coarsely divided (e.g., high or low only), or the range may be finely divided (e.g., a 100 point scale).

Step 804 may include determining whether a low sensitivity has been indicated. Step 808 may include determining whether an intermediate sensitivity has been indicated. Step 812 may include determining whether a high sensitivity has been indicated.

In an illustrative example, referencing flow diagram 800 of FIG. 8, a user may indicate an alarm sensitivity for a physiological parameter such as SpO₂, pulse rate, respiration rate, blood pressure, temperature, or any other suitable physiological parameter, or any combination thereof. An alarm setting for the physiological parameter may be set to an integral threshold if “low” sensitivity is indicated, so that transient excursions are less likely to activate the alarm. Alternatively, an alarm setting for the physiological parameter may be set an alarm threshold if “high” sensitivity is indicated, so that any limit violation will activate the alarm. An alarm setting for the physiological parameter may be set to both an integral threshold and an alarm limit if an intermediate alarm sensitivity between “low” and “high” is indicated, so that small transient deviations do not activate the alarm but larger transient deviations will activate the alarm. Although this example illustrates particular alarm settings, any suitable alarm settings or combinations thereof may be used in accordance with the present disclosure.

In a further illustrative example, referencing flow diagram 800 of FIG. 8, a user may indicate an alarm sensitivity for a physiological parameter such as SpO₂, pulse rate, respiration rate, blood pressure, temperature, or any other suitable physiological parameter, or any combination thereof. An alarm setting for the physiological parameter may be set to a particular integral threshold if “low” sensitivity is indicated, so that transient excursions are not likely to activate the alarm. Alternatively, an alarm setting for the physiological parameter may be set to a lower integral threshold if “high” sensitivity is indicated, so that transient excursions are more likely to activate the alarm. An alarm setting for the physiological parameter may be set to an intermediate integral threshold if an intermediate alarm sensitivity between “low” and “high” is indicated. Although this example illustrates particular alarm settings, any suitable alarm settings or combinations thereof may be used in accordance with the present disclosure.

In a further illustrative example, referencing flow diagram 800 of FIG. 8, a user may indicate an alarm sensitivity for an operating condition metric such as sufficient monitor or sensor power, sensor signal quality, patient movement, or any other suitable operating condition metric, or any combination thereof. An alarm setting for sensor signal quality may be set to a duration threshold of 30 seconds if “low” sensitivity is indicated, so that short intervals of poor signal quality do not activate the alarm. Alternatively, an alarm setting for sensor signal quality may be set to a duration threshold of 10 seconds if “high” sensitivity is indicated, so that short intervals of poor signal quality are more likely (relative to the “low” indication) to activated the alarm. Although this example illustrates particular alarm settings, any suitable alarm settings or combinations thereof may be used in accordance with the present disclosure.

In a further illustrative example, referencing flow diagram 800 of FIG. 8, a user may indicate an alarm sensitivity for an operating condition metric such as a sensor disconnect (e.g., unplugged sensor) or other state change. For example, if “low” sensitivity is indicated, an alarm may be activated after 1 minute of continuous sensor disconnect. Alternatively, if “high” sensitivity is indicated, an alarm may be activated after 10 seconds of continuous sensor disconnect, or at each determination of a sensor disconnect condition. In some embodiments, the volume of an audible alarm when activated may correspond to the indicated alarm sensitivity (e.g., high volume for high sensitivity, low volume for low sensitivity). Although this example illustrates particular alarm settings, any suitable alarm settings or combinations thereof may be used in accordance with the present disclosure.

In a further illustrative example, referencing flow diagram 800 of FIG. 8, a user may indicate an alarm sensitivity for a physiological parameter such as SpO₂, pulse rate, respiration rate, blood pressure, temperature, or any other suitable physiological parameter, or any combination thereof. Alarms may be completely disabled if “low” sensitivity is indicated, so that no alarms will be activated. Alternatively, an alarm setting for the physiological parameter may be an alarm threshold for a particular time duration if “high” sensitivity is indicated, so that any limit violation for longer than the particular duration activates the alarm. Although this example illustrates particular alarm settings, any suitable alarm settings or combinations thereof may be used in accordance with the present disclosure.

In a further illustrative example, referencing flow diagram 800 of FIG. 8, a user may indicate an alarm sensitivity for a combination of physiological parameters such as SpO₂, pulse rate, respiration rate, blood pressure, temperature, or any other suitable physiological parameter. Alarm settings for the combination of physiological parameters may be an alarm limit for a single physiological parameter if “low” sensitivity is indicated, so that a limit violation of a single physiological parameter will activate the alarm. Alternatively, alarm settings for the physiological parameters may be alarm thresholds for each of the physiological parameters if “high” sensitivity is indicated, so that any limit violation activates the alarm. In regards to this example, increased alarm sensitivity includes monitoring an increased number of physiological parameters. Although this example illustrates particular alarm settings, any suitable alarm settings or combinations thereof may be used in accordance with the present disclosure.

It will be understood that the illustrative steps of FIGS. 6-8 may be implemented using any human-readable or machine-readable instructions on any suitable system or apparatus, such as those described herein (e.g., patient monitoring system 10 of FIG. 1).

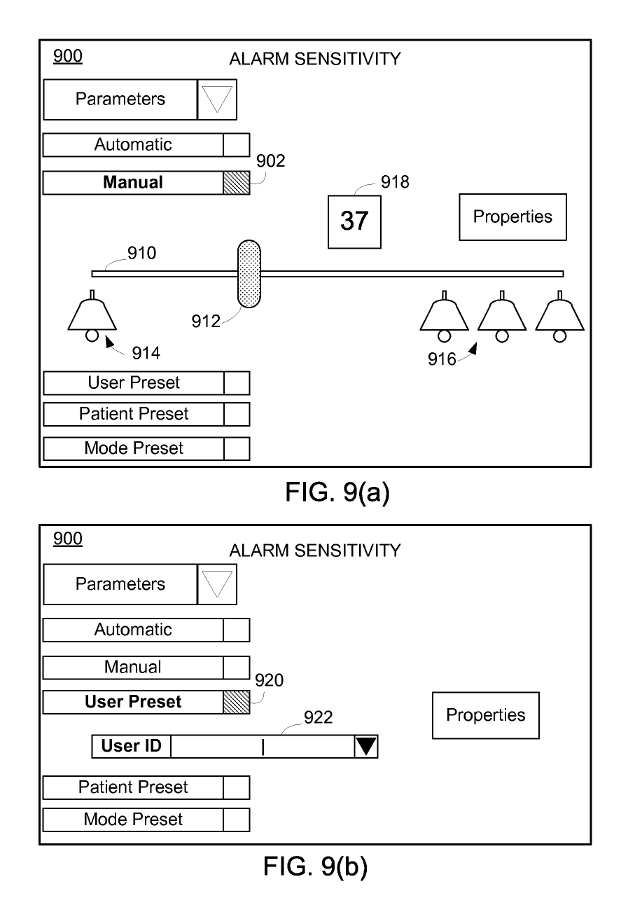

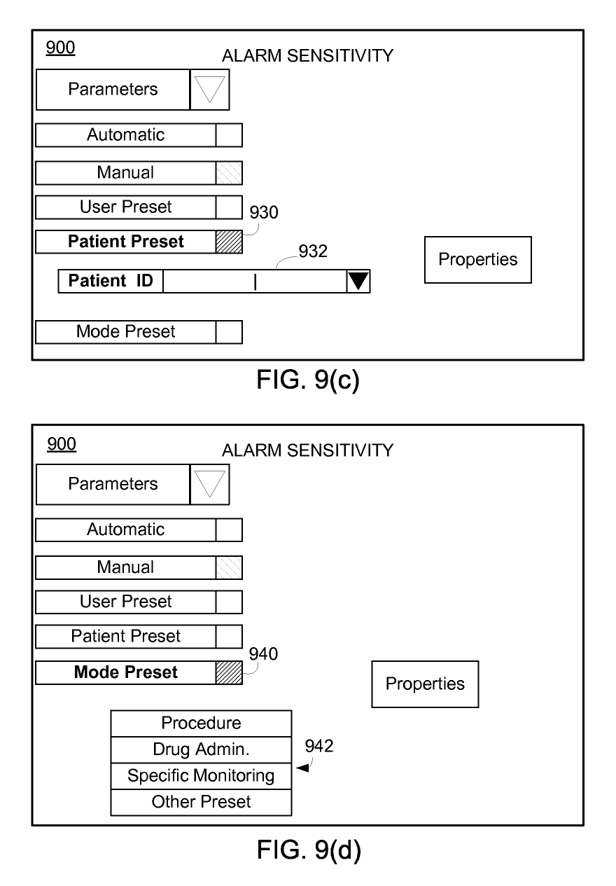

FIGS. 9(a)-9(d) show illustrative indications of alarm sensitivity, in accordance with some embodiments of the present disclosure.

Illustrative display 900 of FIG. 9(a) includes options for indicating alarm sensitivity based on automatic selection (e.g., a default selected by a patient monitor), manual selection (e.g., a user selection), a preset categorized by user (e.g., using a user identification), a preset categorized by patient (e.g., using a patient identification), or a mode preset (e.g., based on a medical procedure). The manual option has been selected as shown by the highlighted “Manual” field 902. It will be understood that illustrative display 900 is used for illustrating some embodiments, and that any suitable options, user interface, display, and any combinations thereof may be used in accordance with the present disclosure.

Slide field 910 may represent a range of sensitivity values varying from low sensitivity, as shown by icon 914, to high sensitivity, as shown by icon 916. In some embodiments, a user may select a desired alarm sensitivity by moving slide 912 along slide field 910 (e.g., an axis) to reach a position corresponding to a desired alarm sensitivity. For example, positional information may be received by a processor of a patient monitoring system which may include the position of slide 912. In some embodiments, a user may enter a sensitivity index (e.g., a number, letter or other suitable character) representing a desired alarm sensitivity into text field 918. In some embodiments, a user may do either of both of moving slide 912 and entering a sensitivity index into text field 918 to indicate a particular alarm sensitivity. In the illustrated example, the number “37” has been entered into text field 918, out of a 1-100 range, indicating an intermediate-to-low sensitivity.

Although shown in FIG. 9(a) as a user input of a desired alarm sensitivity, any suitable indication of alarm sensitivity may be used in accordance with the present disclosure. For example, user identification may be used as shown by field 920 of FIG. 9(b), and may include a name being entered into a suitable text field 922 to recall a preset alarm sensitivity for the user. In a further example, a patient identification as shown by field 930 of FIG. 9(c) may be used, and may include a name being entered into a suitable text field 932 to recall a preset alarm sensitivity for the patient. In a further example, a particular mode may be selected as shown by field 940 of FIG. 9(d). The mode may refer to a medical procedure, drug administration, specific condition, or other preset, as shown by menu 942 to recall a preset alarm sensitivity for the particular mode. A medical procedure identification such as, for example, a surgery type may be selected from menu 942 to recall a preset alarm sensitivity for that surgery type. A drug administration identification such as, for example, a drug name may be selected from menu 942 to recall a preset alarm sensitivity for administration of that drug type. A specific monitoring identification such as, for example, pulse rate monitoring may be selected from menu 942 to recall a preset alarm sensitivity for monitoring a pulse rate. In some embodiments, combinations of user identification, patient identification, mode presets, user selection, or other criteria may be used to indicate an alarm sensitivity. For example, an alarm sensitivity may be selected by a user entering a patient identification and a surgery type to recall a preset alarm sensitivity.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof which are within the spirit of the following claims.

What is claimed is:

1. A method for configuring alarm settings of a patient monitoring system, the method comprising:
receiving a user indication of alarm sensitivity; and
automatically configuring, using a processor, the alarm settings of the patient monitoring system based at least in part on the user indication.

2. The method of claim 1, wherein the user indication comprises a sensitivity index.

3. The method of claim 1, wherein receiving the user indication of alarm sensitivity comprises receiving positional information for a slide bar along an axis, the positional information comprising a position corresponding to a level of alarm sensitivity.

4. The method of claim 1, wherein the configuring the alarm settings further comprises configuring the alarm settings for one or more physiological parameters.

5. The method of claim 4, wherein the one or more physiological parameters comprise a physiological parameter selected from the group consisting of blood oxygen saturation, pulse rate, respiration rate, blood pressure, temperature, and a combination thereof.

6. The method of claim 1, wherein the alarm settings comprise an alarm setting selecting from the group consisting of a limit threshold, a change threshold, an integral threshold, an alarm state, a duration threshold, a number of violations threshold, and a combination thereof.

7. The method of claim 1, wherein the configuring the alarm settings further comprises configuring the alarm settings of one or more operating condition metrics.

8. The method of claim 1, wherein the configuring the alarm settings further comprises configuring the alarm settings of one or more signal metrics.

9. The method of claim 1, wherein the receiving the user indication comprises receiving a user indication selected from the group consisting of a patient identification, a user identification, a medical procedure identification, a drug administration identification, a default alarm sensitivity identification, and a combination thereof.

10. The method of claim 1, further comprising recalling stored alarm settings based at least in part on the user indication, and wherein the configuring the alarm settings of the patient monitoring system is further based at least in part on the stored alarm settings.

11. A patient monitoring system comprising:
a user input interface configured to receive a user indication; and
a processor coupled to the user input interface, the processor configured to configure the alarm settings of the patient monitoring system based at least in part on the user indication.

12. The system of claim 11, wherein the user indication comprises a sensitivity index.

13. The system of claim 11, wherein the receiving the user indication of alarm sensitivity comprises receiving positional information for a slide bar along an axis, the positional information comprising a position corresponding to a level of alarm sensitivity.

14. The system of claim 11, wherein the processor is further configured to configure the alarm settings of one or more physiological parameters.

15. The system of claim 14, wherein the one or more physiological parameters comprise a physiological parameter selected from the group consisting of blood oxygen saturation, pulse rate, respiration rate, blood pressure, temperature, and a combination thereof.

16. The system of claim 11, wherein the alarm settings comprise an alarm setting selecting from the group consisting of a limit threshold, a change threshold, an integral threshold, an alarm state, a duration threshold, a number of violations threshold, and a combination thereof.

17. The system of claim 11, wherein the processor is further configured to configure the alarm settings of one or more operating condition metrics.

18. The system of claim 11, wherein the processor is further configured to configure the alarm settings of one or more signal metrics.

19. The system of claim 11, wherein the user indication comprises a user indication selected from the group consisting of a patient identification, a user identification, a medical procedure identification, a drug administration identification, a default alarm sensitivity identification, and a combination thereof.

20. The system of claim 11, further comprising:
memory configured to store alarm settings,
wherein the processor is further configured to recall stored alarm settings based at least in part on the user indication, and
wherein the processor is further configured to configure the alarm settings of the patient monitoring system is further based at least in part on the stored alarm settings.
